(19) United States
(12) Patent Application Publication
(10) Pub. No.: US 2019/0021992 A1
(43) Pub. Date: Jan. 24, 2019

(54) NEW IMPROVED COMPOSITION COMPRISING AT LEAST ONE CADOTRIL

(71) Applicant: Johnson & Johnson Consumer Inc., Skillman, NJ (US)

(72) Inventors: SALIH MUHSIN MUHAMMED, Hyllinge (SE); KATARINA LINDELL, ESLOV (SE); ANDREAS HUGERTH, BJARRED (SE)

(21) Appl. No.: 16/068,720

(22) PCT Filed: Jan. 9, 2017
(86) PCT No.: PCT/US2017/012665

§ 371 Date: Jul. 9, 2018

(30) Foreign Application Priority Data
Jan. 13, 2016 (SE) 1650033-2

(51) Int. Cl. A61K 9/00; A61K 31/216; A61K 47/10; A61K 47/14; A61K 31/80

(52) U.S. Cl. CPC A61K 9/0056; A61K 31/216; A61K 31/80; A61K 47/14; A61K 47/10

(57) ABSTRACT

The invention relates to a semi-solid composition comprising at least one cadotril and at least one liquid-lipid and/or polyol and/or glycerol and/or propylene glycol excipient, a dose unit comprising the semi-solid composition as well as use and a method of treating a subject suffering from a disease or disorder in the gastro intestinal tract.

### FIELD OF INVENTION

The invention relates to a semi-solid composition comprising at least one cadotril and at least one liquid-lipid and/or polyol and/or glycerol and/or propylene glycol excipient, a dose unit comprising the semi-solid composition as well as use and a method of treating a subject suffering from a disease or disorder in the gastro intestinal tract.

### BACKGROUND OF INVENTION

Diarrhea is an intestinal disorder that is characterized by an increase in the frequency of watery bowel movements. It may result from a variety of causes including bacteria or viral induced diarrhea. Food intolerance caused by allergy or the consumption of foods such as fatty or spicy foods may result in diarrhea. Food poisoning may also lead to diarrhea. In some instances, diarrhea may be a symptom of other conditions and diseases. One example is the irritable bowel syndrome (IBS). A patient with IBS typically presents clinically with one of three variants: i) chronic abdominal pain and constipation (also known as spastic colitis); ii) chronic intermittent diarrhea, often without pain; or iii) both features, in an alternating cycle of constipation and diarrhea.

Diarrhea is symptomatic of an intestinal or other bodily function disorder. Various prescription and nonprescription products can be taken for relief. However, many of these products provide relief with some side effects.

Cadotril compounds, such as Racecadotril is used in the treatment of diarrhea. It reduces (i) hypersecretion of water and electrolytes into the intestinal lumen, (ii) the incidence and duration of acute diarrhea and (iii) diarrhea-associated symptoms.

US2015/0342882 discloses the use of liquid formulation comprising cadotril compounds, but is silent about semi-solid formulations.

Tran & Wang, 2014, Front Chem Sci. Eng 8(2):225-232 is a review article wherein different semi-solid materials are described, such as polysaccharides, lipid based systems, cellulose derivatives, starch and amylose, chitosan, alginate and carbomers.

However, there are some draw backs with the existing formulations and there is a need for new improved ones.

Simethicone is an orally administered anti-foaming agent used to reduce bloating, discomfort or pain caused by excessive gas, mainly swallowed air, with small amounts of hydrogen and methane in the stomach or intestines.

There is a need for new products containing either cadotril or a combination of cadotril and simethicone to be able to provide new products on the market which aim at helping consumers suffering from a disorder or disease within the gastro intestinal tract.

### SUMMARY OF THE INVENTION

The invention relates to the development of new improved Cadotril compositions, such as Racecadotril alone or in combination with Simethicone. Such compositions have improved properties compared to present compositions including improved uptake, bioavailability and/or controlling release, such as sustained or delayed release. By governing and controlling the release it is possible to achieve and intended profile of the pharmaceutical agent, such as a faster or longer effect depending on the intended use. By the use of few and less toxic excipients it is as well possible to obtain a composition that is not harmful for the subject.

In a first aspect the invention relates to a semi-solid composition comprising at least one cadotril, such as racecadotril and at least one liquid-lipid and/or polyol and/or glycerol and/or propylene glycol, whereby the invented semi-solid composition solves one or more of the above defined problems, such as influencing the effect on onset as well as duration.

It has surprisingly been found that such a composition creates a special crystalline structure during manufacturing, which will give rise to a high surface area, which is substantially free from water and which reduces the stability problem with the active ingredients. Formulating racecadotril in an oil base or other suitable water free liquids as mentioned below provides protection from hydrolysis in addition to improved absorption, rapid onset of action and increased bioavailability. Increased bioavailability permits the use of lower doses of racecadotril to achieve an efficacious therapeutic window, thus minimizing potential side effects associated with use of racecadotril.

One example how to make the cadotril, such as racecadotril may be by mixing racecadotril with one or more suitable liquid solvent excipients, heating the mixture and mixing to dissolve cadotril, such as racecadotril and to obtain a homogenized liquid mixture. The mixture is allowed to cool down with continuous mixing. Due to simultaneous mixing and cooling, cadotril such as racecadotril crystalize to fine crystals. The produced mixture show semisolid consistency which is believed being attributed to the friction forces between the fine crystals as well as crystals interactions with the other components.

In a second aspect the invention relates to a semi-solid composition comprising at least one cadotril, such as racecadotril, simethicone and at least one liquid-lipid and/or polyol or glycerol or propylene glycol mixture thereof, which may be substantially free from surfactants, such as composition will for the first time enable the possibility to facilitate the administration of both ingredients in one composition.

In a third aspect the invention relates to a dose unit comprising either cadotril, such as racecadotril or cadotril, such as racecadotril together with simethicone. Such doses will give rise to the possibility to provide higher concentrations/doses of the cadotril, such as racecadotril in the doses compared to what is possible today as well as due to the increased bioavailability decrease the concentration/dose. By providing a prolonged release profile it will secure that plasma levels will be kept within the effective concentration range for a longer period of time. Such prolonged release plasma profile makes it possible to reduce dosing frequency as well as having once or twice daily dosing instead of three times daily dosing product available on the market. In addition, the prolonged release may help to avoid too high plasma concentration, which could lead to increased risk for adverse or unwanted effects. There is no product available on the market today providing the mixer of cadotril, such as racecadotril and simethicone and which solves the above identified problems. By the use of the specific crystalline structure of racecadotril is possible to load a high dose and still have a dose unit is possible to swallow. However, part of the active ingredient in the crystalline network, about 1.5 wt % or less is in the soluble state and thus the soluble part will give rise to a fast release and thereby a fast effect with a low dose, when medium chain triglyceride (MCT) is used as the liquid excipient.

Finally, the invention relates to a method of using the semi-solid composition(s) defined or the dose unit defined above for the treatment of a subject suffering from a disease or disorder in the gastro intestinal tract, wherein the semi-solid could be co-administrated with one or more dietary fiber products.

### BRIEF DESCRIPTION OF THE DRAWINGS

FIG. 1: Mean thiorphan plasma concentrations time curve following per oral administration of racecadotril semisolid formulations, alone or in combination with Simethicone at a dose of 20 mg/kg in male beagle dogs study. A granulate powder from commercial capsule formulation, racecadotril (Vaprino100 mg®) was used as a reference formulation for comparison. Each dog (n=12) was administered (P.O.) two capsules (equivalent to 200 mg racecadotril).

FIG. 2: Mean thiorphan plasma concentrations time curve in rat (n=3) following per oral administration of semisolid formulation of racecadotril in combination with Simethicone at a dose of 20 mg/kg racecadotril and simethicone 25 mg/kg [Formulation nr. 8 (example 2)]. A thiorphan i.v. (dose 13.4 mg/kg) was used as a reference formulation for comparison.

FIG. 3: Mean thiorphan plasma concentrations time curve in rat (n=5) following per oral administration of semisolid formulation of racecadotril in combination with Simethicone [Formulation nr. 9, 10 (example 2)] at a dose of 20 mg/kg racecadotril and simethicone 25 mg/kg. Racecadotril (Vaprino) at a dose of 20 mg/kg was used as a reference formulation for comparison.

FIG. 4: Mean thiorphan plasma concentrations time curve in rat (n=5) following per oral administration of semisolid formulation of racecadotril in combination with Simethicone [Formulation nr. 14, 15 and 16 (example 2)] at a dose of 20 mg/kg racecadotril and simethicone 25 mg/kg. Racecadotril (Vaprino) at a dose of 20 mg/kg was used as a reference formulation for comparison.

FIG. 5: Mean thiorphan plasma concentrations time curve in rat following per oral administration of racecadotril semisolid formulations, alone [Formulation nr. 4 (example 2), (n=4)] or in combination with Simethicone [Formulation nr. 11 (example 2), (n=5)] at a dose of 20 mg/kg at a dose of 20 mg/kg racecadotril and simethicone 25 mg/kg. Racecadotril (Vaprino) at a dose of 20 mg/kg was used as a reference formulation (n=5) for comparison.

### DETAILED DESCRIPTION OF EMBODIMENTS OF THE INVENTION

### Definitions

In the context of the present application and invention the following definitions apply:

The term Racecadotril, chemically known as benzyl N-[3-(acetylthio)-2 benzylpropanoyl] glycinate is an anti-diarrheal drug which acts as a peripherally acting enkephalinase inhibitor. It has an anti-secretory effect and used to treat diarrhoea. It reduces the secretion of water and electrolytes into the intestine. Racecadotril is a prodrug and it is hydrolysed to its active metabolite, thiorphan following intravenous or oral administration.

The term semisolid is intended to mean the physical term for something that lies along the boundary between a solid and a liquid. While similar to a solid in some respects, in that semisolids can support their own weight and hold their shapes, a semisolid also shares some properties of liquids, such as conforming in shape to something applying pressure to it and the ability to flow under pressure. The words semisolid, quasisolid and semiliquid all mean exactly the same thing.

The term Medium Chain Triglyceride(s) (MCTs) is/are intended to mean triglycerides whose fatty acids have an aliphatic tail of 6-12 carbon atoms. The fatty acids found in MCTs are called medium-chain fatty acids (MCFAs). Like all triglycerides, MCTs are composed of a glycerol backbone and three fatty acids. In the case of MCTs, 2 or 3 of the fatty acid chains attached to glycerol are medium-chain in length. Examples includes, hexanoic acid (C6:0, common name caproic acid), octanoic acid (C8:0, common name caprylic acid), and decanoic acid (C10:0, common name capric acid) as well as dodecanoic acid (C12:0, common name lauric acid).

The term “substantially free from water or free from water” is intended to mean that the content of water present in the semi-solid composition is less than about 2 wt. % based on the total wt. % of the semi-solid composition, such as less than 1.5, 1, 0.5, 0.4, 0.3, 0.2 or less than 0.1 or totally free from water, i.e., 0 wt % based on the total wt. % of the semi-solid composition.

The term “substantially free from non-ionic surfactants or free from non-ionic surfactants” is intended to mean that the content of the non-ionic surfactant present in the semi-solid composition is less than about 2 wt. % based on the total wt. % of the semi-solid composition, such as less than 1.5, 1, 0.5, 0.4, 0.3, 0.2 or less than 0.1 or totally free from surfactant, i.e., 0 wt. % based on the total wt. % of the semi-solid composition. The definition of a non-ionic surfactant is well-known for a person skilled in the art as being compounds that lower the surface tension (or interfacial tension) between two liquids or between a liquid and a solid. Non-ionic surfactants are amphiphilic molecules that have both a hydrophobic group non-polar “tail” and a hydrophilic group polar but uncharged “head”. Prominent among these are the fatty alcohols, cetyl alcohol, stearyl alcohol, and cetostearyl alcohol (consisting predominantly of cetyl and stearyl alcohols), and oleyl alcohol.

The term “% w/w” is intended to mean the percentage of an ingredient(s)/the total percentage by weight of the composition (100%).

The term “bioavailability” is intended to mean, the rate and extent to which the active substance or active moiety is absorbed from a pharmaceutical form and becomes available at the site of action. Bioavailability of oral Racecadotril is assessed by monitoring concentrations of Racecadotril active metabolite (thiorphan) in the general circulation.

A “dosage”, “dosage form”, “dose unit” or “dose” as used herein means the amount of a pharmaceutical formulation comprising therapeutically active agent(s) administered at a time. “Dosage”, “dosage form”, “dose unit” or “dose” includes administration of one or more units of pharmaceutical formulation administered at the same time.

### The Semi-Solid Composition

The invention relates to a semi-solid composition comprising one or more active ingredients and at least one liquid-lipid and/or polyol or glycerol or propylene glycol or a mixture thereof. The liquid-lipid excipient is at least one medium chain triglyceride or at least one oil containing medium chain triglycerides or a mixture thereof.

The semi-solid composition may be substantially free from non-ionic surfactants and/or being substantially free from water as defined above. By not utilizing any non-ionic surfactant, there will be no problem with unpleasant taste, irritation or toxic effect will occur which is common upon using surfactants.

The at least one medium chain triglyceride (MCT), i.e., an ester of glycerol and a medium chain fatty acid or a natural oil containing medium chain fatty acids, wherein the medium chain fatty acids are selected from the group consisting of caprylic acid (C8), caproic (C6) acid, capric acid (C10), lauric acid (C12) or mixture thereof. One example being that the medium chain triglyceride is an ester of glycerol and one or more medium chain fatty acids being caprylic or capric acid or a mixture thereof. Other examples are found in table 1.

The at least one liquid-lipid and/or polyol or glycerol or propylene glycol excipient or mixtures thereof is/are present in an amount of from about 15% w/w to about 95% w/w of the total amount, such as from about 20% w/w to about 90% w/w, from about 25% w/w to about 90% w/w from about 40% w/w to about 80% w/w, from about 60% w/w to about 70% w/w, from about 80% w/w to about 90% w/w of the total amount of the composition, such as 75% w/w, about 80% w/w or about 85% w/w.

The Medium chain fatty acid (MCFA), is one or more medium chain fatty acids selected from the group consisting of caprylic acid (C8), caproic (C6) acid, capric acid (C10), lauric acid and esters of caprylic acid (C8), caproic (C6) acid, capric acid (C10), lauric acid. The MCFA may be a mixture of caprylic and capric acid. The amount of the MCFA's in a MCT may be C6<2.0%, C8 about 50-80%, C10 about 20-50%. One example is C12<3.0% and C14<1.0% and the total amount of C8 and C10 up to 95% and the water content <0.2%.

Examples of MCTs presented on the market today are shown in table 1 below.

TABLE 1
Examples of commercial MCT.

| | | Chemical description/INCI | |
|---|---|---|---|
| Product-Trade Mark | Supplier | name | Listed in |
| Crodamol GTCC- | Croda | Caprylic/Capric | Triglycerides, |
| LQ-(MV) | | Triglyceride | Medium-Chain PhEur; |
| Old Trade Mark: | | | Medium-Chain |
| Estasan GT8-60 | | | Triglycerides NF |
| 3575 | | | (Caprylic/Capric |
| | | | Triglycerides |
| MIGLYOL 812 | Sasol | Caprylic/Capric Triglyceride | Ph. Eur., USP-NF, |
| | | | JPE, DMF |
| LABRAFAC | Gattefossé | Triglycerides medium-chain | DMF |
| LIPOPHILE WL | | EP/Medium-chain | USP/NF |
| 1349 | | triglycerides NF/Medium | EP |
| | | chain fatty acid triglyceride | JP/JPE |
| | | JPE | |
| NEOBEE ® M-5 | STEPAN | Captrin, Medium Chain | Complies with the |
| | | Triglycerides, Caprylic/Capric | specifications for |
| | | Triglycerides or Glyceryl | Medium Chain |
| | | Tri(caprylate/caprate). | Triglycerides of the |
| | | | National formulary as |
| | | | published by the U.S. |
| | | | Pharmacopoeia (USP |
| | | | 27/NF 22) and with EP |
| | | | and JPE. NEOBEE M- |
| | | | 5 is Kosher and Halal |
| | | | Certified. NEOBEE |
| | | | M-5 has a Type IV |
| | | | Drug Master File |
| | | | (DMF) available. |
| CAPTEX ® 355 | Abitec | Glycerol Tricaprylate/Caprate, | Meets current |
| | Corporation | Medium Chain Triglyceride | European |
| | | (MCT); Caprylic/Capric | Pharmacopoeia for |
| | | Triglyceride; | Triglycerides, |
| | | Octanoic/Decanoic Acid, | Medium-Chain, United |
| | | Triglyceride | States |
| | | | Pharmacopeia/National |
| | | | Formulary for |
| | | | Medium-Chain |
| | | | Triglyerides and |
| | | | Japanese |
| | | | Pharmaceutical |
| | | | Excipients |
| | | | monographs for |
| | | | Medium Chain (Fatty |
| | | | Acid) Triglycerides. |

The MCTs shown in table 1 above can be purchased from the following companies. Miglyol 812 from SASOL GmbH, CRODAMOL GTCC from Croda, or Neobees M-5 oil from Stepan and LABRAFAC LiPOPHILE WL 1349 from Gattefossé.

Examples of natural oils that could be used are all natural oils comprising MCTs, such as coconut or palm kernel oils.

In a first embodiment the invention relates to a semi-solid composition comprising at least one cadotril and at least one liquid-lipid and/or polyol or glycerol or propylene glycol excipient. Examples and definition of the excipient being found above.

Example of cadotril includes racecadotril, dexecadotril and ecadotril or mixtures thereof. In the examples below racecadotril has been used. Cadotril(s) is/are present in an amount from about 5 w/w to about 50%, such as w/w 10% w/w to about 30% w/w, such as about 15% w/w, about 20% w/w or about 25% w/w. In one example the cadotril coexist in a dissolved and solid state present within the semi-solid composition.

The amount of the different ingredients present within the semi-solid composition may vary depending on which other components should be included.

In a second embodiment the invention relates to a semi-solid composition comprising cadotril and simethicone and at least one liquid-lipid and/or polyol or glycerol or propylene glycol excipient or mixtures thereof. Examples and definition of the excipient being found above. Example of cadotril includes racecadotril, dexecadotril and ecadotril or mixtures thereof. In the examples below racecadotril has been used. The amounts of cadotril being defined above.

Simethicone may be available from DOW CORNING® Q7-2243 LVA, SIMETHICONE USP, or DOW CORNING Antifoam M.

Simethicone is present in an amount of about 5% w/w to about 75% w/w, about 5% w/w to about 70% w/w. about 5 w/w to about 60% w/w 5% w/w to about 35% w/w 10% w/w to about 35% w/w, such as about 18.75% w/w, about 25% w/w or about 31.25% w/w.

The embodiments may further comprise one or more ingredient(s) selected from the list consisting of coloring agents, antioxidants, flavoring agents, sweeteners, thickeners, emulsifiers, excipients, preservatives and gelling agents.

Additionally, the composition may comprise one or more fibres, such as dietary fibre which consists of non-starch polysaccharides such as arabinoxylans, cellulose, and many other plant components such as resistant starch, resistant dextrins, inulin, lignin, waxes, chitins, pectins, beta-glucans, and oligosaccharides and other types of carbohydrate that the body can't digest and simply passes through the entire digestive tract. Generally, fibres comes from plant foods: fruits, vegetables, grains, nuts, and legumes such fibre are classified as soluble fibres such as psyllium fibres, others are classified as insoluble fibres like those found in the seeds and skins of fruit as well as whole-wheat bread and brown rice.

The first and second embodiment may further comprise an additional active ingredient. The additional active ingredient may be, a digestive health active ingredient, for example, laxatives, antacids, proton pump inhibitors, anti-gas agents, antiemetics, H2 blockers, a second antidiarrheal agent, and the like. Examples of additional agents includes loperamide, α-galactosidase enzyme, calcium carbonate, aluminum hydroxide and magnesium hydroxide.

### A Dose Unit

The invention also relates to a dose unit, wherein the dose unit comprises either the semi-solid composition according to the first embodiment defined above and/or the semi-solid composition according to the second embodiment defined above. Cadotril(s), such as racecadotril, dexecadotril and ecadotril or mixtures thereof is/are present in an amount from about 5 mg to about 200 mg, such as about 5 mg or about 100 mg and simethicone is present in an amount from about 50 to about 1500 mg, such as about 50 to about 1000 mg, such as about 50 to about 500 mg, such as about 50 to about 100 mg, such as about 2 mg to about 150 mg, such as about 6.25 or about 125 mg. The dosage form may be a tablet or capsule.

### Use and Method of Treatment

Further the invention relates to the semi-solid composition as defined above or the dose unit defined above for the treatment of a subject suffering from a disease or disorder in the gastro intestinal tract, such as IBS, such as diarrhea and/or constipation and/or bloating, discomfort or pain caused by excessive gas.

Finally the invention relates to a method of treatment of a subject suffering from a disease or disorder in the gastro intestinal tract, such as IBS, such as diarrhea and/or constipation and/or bloating, discomfort or pain caused by excessive gas.

Following examples are intended to illustrate, but not to limit, the invention in any manner, shape, or form, either explicitly or implicitly.

### Examples 1: Preparation of Bulk Semi-Solid Formulation to be Used in the Examples Below

1. Racecadotril is weighed in a scintillation vial.
2. The MCT (obtained from Croda) is weighed and added in the same vial.
3. The mixture is heated in a water bath at 80° C. (the vial is closed to avoid direct contact with water or water vapor) and mixed using stirred using vortex or homogenizer to dissolve racecadotril and achieve a clear homogenous solution.
4. Cooling down at room temperature with continuous mixing in order to obtain a semi-solid formulation.

Simethicone can be added either before heating and mixing or to be mixed with the semisolid at the end.

### Example 2: Formulations

| Formulation nr. | |
|---|---|
| | Composition (% w/w) |
| 1 | Racecadotril 15%, MCT 85% |
| 2 | Racecadotril 20%, MCT 80% |
| 3 | Racecadotril 25%, MCT 75% |
| 4 | Racecadotril 15%, propylene glycol (PG) 85% |
| 5 | Racecadotril 20%, propylene glycol (PG) 80% |
| 6 | Racecadotril 25%, propylene glycol (PG) 75% |
| 7 | Racecadotril 30%, propylene glycol (PG) 70% |
| | Composition (%) |
| 8 | Racecadotril 15%, Simethicone 18.75%, MCT |
| | 66.25% |
| 9 | Racecadotril 20%, Simethicone 25% in MCT 55% |
| 10 | Racecadotril 25%, Simethicone 31.25% in MCT |
| | 43.75% |
| 11 | Racecadotril 15%, Simethicone 18.75%, PG 66.25% |
| 12 | Racecadotril 20%, Simethicone 25%, PG 55% |
| 13 | Racecadotril 25%, Simethicone 31.25%, PG 43.75% |
| 14 | Racecadotril 10%, Simethicone 20%, MCT 70% |
| 15 | Racecadotril 10%, Simethicone 40%, MCT 50% |
| 16 | Racecadotril 10%, Simethicone 60%, MCT 30% |

### Example 3: Stability Studies

Formulations nr. 1, 8 and 9 (example 2) were included in stability studies and have demonstrated stability up to 3 months at 25, 40 and 50° C. with 98-100% nominal dose remaining.

### Example 4: Oral Administration Crossover Pharmacokinetic Studies in Male Beagle Dogs

Formulation nr. 1, 8 and 9 (example 2) were administered dogs p.o. at doses expected to be pharmacologically effective. For estimation of the bioavailability, one group of dogs was given the reference product, a granulate powder from commercial capsule formulation, racecadotril (Vaprino® 100 mg). Blood was drawn at specified time points and plasma concentration of thiorphan was assessed.

### Study Parameters:

Animals: male beagle dogs (N=12) of similar age (2-5.5 yrs) and body weight (9-12 kg) were selected to receive each formulation.

### Animal Preparation:

Each dog was injected intramuscularly (IM) with pentagastrin solution ˜30 mins prior to dosing to maintain the stomach pH ˜1.2, which is similar to human.
Each dog was administered (P.O.) two capsules (equivalent to 200 mg racecadotril) followed by a dosing flush of 100 mL of sterile water.
The washout period between dosing each formulation was 5 days. Blood samples were collected at pre-determined time points (2, 5, 15, 30, 45 min, 1, 1.5, 2, 3, 4, 6 and 8 hours) and centrifuged at 4° C. with 3000×g for 5 mins.
Plasma samples were transferred into appropriate storage vials and treated with the derivatizing reagent 2-bromo-3-methoxyacetophenone (BMP, 0.5 M in acetonitrile) for 10 mins prior to being immediately frozen on dry ice to stabilize the thiorphan.
Plasma samples were then analyzed by LC-MS/MS.
Pharmacokinetic parameters (i.e., AUC, Cₘₐₓ, Tₘₐₓ T_{1/2}, Kel, MRT) were calculated with WinNonlin® software using a non-compartmental model.

### Results:

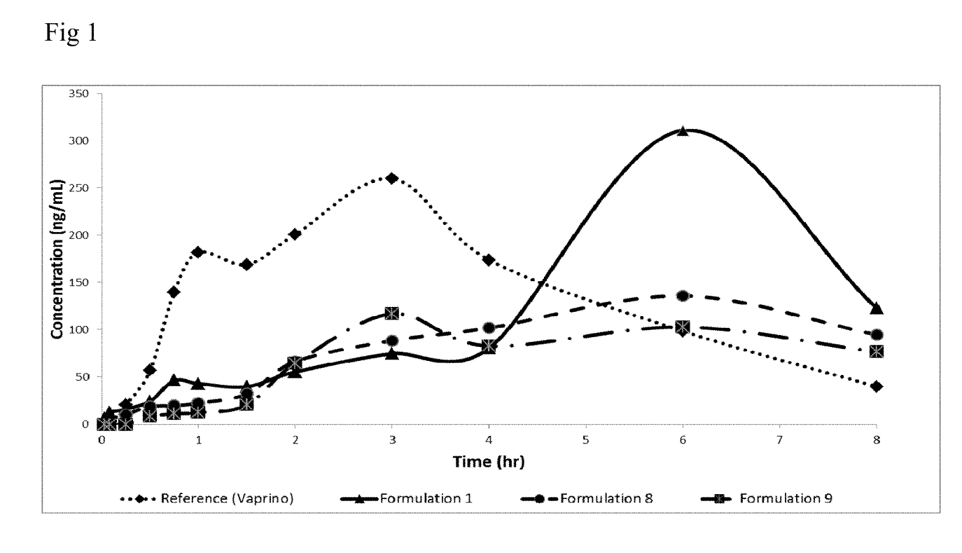

FIG. 1 shows the mean thiorphan plasma concentrations time curve following per oral administration of racecadotril formulations, alone or in combination with Simethicone at a dose of 20 mg/kg in male beagle dogs study. A granulate powder from commercial capsule formulation, racecadotril (Vaprino100 mg®) was used as a reference formulation for comparison.

The results of calculation for pharmacokinetic parameters are shown in tables (2, 3, 4 and 5). Prolonged absorption profile was observed in term Cₘₐₓ and Tₘₐₓ. For formulations nr1, 8 and 9 (example 2) Cₘₐₓ was delayed for as long as 6 hours as compared to reference where Tₘₐₓ was approximately 3 hours.

TABLE 2
Individual and Average Pharmacokinetic Parameters for Thiorphan After Oral Administration of racecadotril reference
formulation (Vaprino100mg ®) in Male Beagle Dogs (200 mg Racecadotril/dog)
Racecadotril reference formulation (Vaprino100mg ®)

| | Dog # | | | | | | | | | | | | | |
|---|---|---|---|---|---|---|---|---|---|---|---|---|---|---|
| Time (hr) | 146 | 147 | 148 | 149 | 150 | 151 | 152 | 153 | 154 | 155 | 156 | 157 | Mean | SD |
| Animal Weight | 11.9 | 11.7 | 9.4 | 8.4 | 8.4 | 9.2 | 8.6 | 8.6 | 9.6 | 8.4 | 10.0 | 10.0 | 9.5 | 1.2 |
| (kg) | | | | | | | | | | | | | | |
| Dose (mg/kg) | 16.8 | 17.1 | 21.3 | 23.8 | 23.8 | 21.7 | 23.3 | 23.3 | 20.8 | 23.8 | 20.0 | 20.0 | 21.3 | 2.5 |
| Cₘₐₓ (ng/mL) | 366 | 432 | 328 | 478 | 152 | 273 | 303 | 499 | 168 | 10.9 | 334 | 356 | 308 | 142 |
| tₘₐₓ (hr) | 2.0 | 1.0 | 3.0 | 3.0 | 3.0 | 1.0 | 1.5 | 3.0 | 1.0 | 4.0 | 3.0 | 2.0 | 2.3 | 1.0 |
| t_{1/2} (hr) | ND² | 1.21 | 1.07 | 3.41 | 2.26 | ND² | ND² | 1.93 | 1.55 | ND³ | 1.28 | ND² | 1.81 | 0.818 |
| MRTₗₐₛₜ (hr) | 2.84 | 2.69 | 3.21 | 3.31 | 3.48 | 3.28 | 3.76 | 4.18 | 3.22 | 2.03 | 3.92 | 3.30 | 3.27 | 0.571 |
| AUCₗₐₛₜ | 991 | 1431 | 1354 | 1241 | 513 | 1020 | 1340 | 1836 | 661 | 21.4 | 1302 | 1126 | 1070 | 481 |
| (hr · ng/mL) | | | | | | | | | | | | | | |
| AUC_{∞} | ND² | 1462 | 1386 | 1520 | 595 | ND² | ND² | 2068 | 702 | ND³ | 1368 | ND² | 1300 | 505 |
| (hr · ng/mL) | | | | | | | | | | | | | | |
| Dose- | | | | | | | | | | | | | | |
| normalized | | | | | | | | | | | | | | |
| Values¹ | | | | | | | | | | | | | | |
| AUCₗₐₛₜ | 59.0 | 83.7 | 63.5 | 52.2 | 21.5 | 47.0 | 57.5 | 78.8 | 31.8 | 0.899 | 65.1 | 56.3 | 51.4 | 23.5 |
| (hr · kg · ng/ | | | | | | | | | | | | | | |
| mL/mg) | | | | | | | | | | | | | | |
| AUC_{∞} | ND² | 85.5 | 65.1 | 63.9 | 25.0 | ND² | ND² | 89 | 33.76 | ND³ | 68.4 | ND² | 61.5 | 24.1 |
| (hr · kg · ng/ | | | | | | | | | | | | | | |
| mL/mg) | | | | | | | | | | | | | | |

Cₘₐₓ: Maximum plasma concentration;
tₘₐₓ: Time of maximum plasma concentration;
t_{1/2}: half-life, data points used for half-life determination are in bold in the respective plasma concentration table;
MRT: mean residence time;
AUCₗₐₛₜ: Area Under the Curve, calculated to the last observable time point;
AUC_{∞}: Area Under the Curve, extrapolated to infinity;
ND: Not Determined;
¹Dose-normalized by dividing the parameter by the dose of racecadotril in mg/kg;
²not determined because the terminal elimination phase had an R² of less than 0.85;
³not determined because of a lack of quantifiable data points trailing the Cₘₐₓ.

TABLE 3
Individual and Average Pharmacokinetic Parameters for Thiorphan After Oral Administration
of Formulation nr. 1 (example 2) in Male Beagle Dogs (200 mg Racecadotril/dog)
Formulation nr. 1 (example 2)

| | Dog # | | | | | | | | | | | | | |
|---|---|---|---|---|---|---|---|---|---|---|---|---|---|---|
| Time (hr) | 146 | 147 | 148 | 149 | 150 | 151 | 152 | 153 | 154 | 155 | 156 | 157 | Mean | SD |
| Animal Weight | 10.4 | 10.9 | 9.1 | 8.5 | 7.7 | 9.8 | 8.7 | 8.6 | 9.5 | 8.6 | 10.2 | 10.2 | 9.4 | 1.0 |
| (kg) | | | | | | | | | | | | | | |
| Dose (mg/kg) | 19.2 | 18.3 | 22.0 | 23.5 | 26.0 | 20.4 | 23.0 | 23.3 | 21.1 | 23.3 | 19.6 | 19.6 | 21.6 | 2.3 |
| Cₘₐₓ (ng/mL) | 245 | 232 | 69.1 | 243 | 188 | 141 | 206 | 1340* | 120 | 810 | 141 | 10.1* | 240 | 209 |
| tₘₐₓ (hr) | 0.75 | 3.0 | 1.0 | 6.0 | 4.0 | 8.0 | 6.0 | 6.0* | 8.0 | 6.0 | 6.0 | 0.25* | 4.9 | 2.3 |
| t_{1/2} (hr) | ND² | ND² | ND² | ND² | ND² | ND² | ND² | ND² | ND² | ND² | ND² | ND³ | ND | ND |
| MRTₗₐₛₜ (hr) | 3.93 | 4.90 | 4.31 | 5.60 | 5.19 | 4.82 | 5.54 | 5.85* | 4.88 | 5.94 | 5.03 | ND³ | 5.01 | 0.601 |
| AUCₗₐₛₜ | 653 | 1135 | 359 | 584 | 759 | 666 | 788 | 3040* | 661 | 2002 | 770 | ND³ | 838 | 453 |
| (hr · ng/mL) | | | | | | | | | | | | | | |
| AUC_{∞} | ND² | ND² | ND² | ND² | ND² | ND² | ND² | ND² | ND² | ND² | ND² | ND³ | ND | ND |
| (hr · ng/mL) | | | | | | | | | | | | | | |
| Dose- | | | | | | | | | | | | | | |
| normalized | | | | | | | | | | | | | | |
| Values¹ | | | | | | | | | | | | | | |
| AUCₗₐₛₜ | 34.0 | 62.0 | 16.3 | 24.9 | 29.2 | 32.7 | 34.3 | 130* | 31.3 | 85.9 | 39.3 | ND³ | 39.0 | 20.2 |
| (hr · kg · ng/ | | | | | | | | | | | | | | |
| mL/mg) | | | | | | | | | | | | | | |
| AUC_{∞} | ND² | ND² | ND² | ND² | ND² | ND² | ND² | ND² | ND² | ND² | ND² | ND³ | ND | ND |
| (hr · kg · ng/ | | | | | | | | | | | | | | |
| mL/mg) | | | | | | | | | | | | | | |

Cₘₐₓ: Maximum plasma concentration;
tₘₐₓ: Time of maximum plasma concentration;
t_{1/2}: half-life, data points used for half-life determination are in bold in the respective plasma concentration table;
MRT: mean residence time;
AUCₗₐₛₜ: Area Under the Curve, calculated to the last observable time point;
²not determined because the terminal elimination phase was not observed;
⁴not determined due to a lack of quantifiable data points;
*value removed as an outlier, see Appendix E.

TABLE 4
Individual and Average Pharmacokinetic Parameters for Thiorphan After Oral Administration
of Formulation nr. 8 (example 2) in Male Beagle Dogs (200 mg Racecadotril/dog)
Formulation nr. 8 (example 2)

| | Dog # | | | | | | | | | | | | | |
|---|---|---|---|---|---|---|---|---|---|---|---|---|---|---|
| Time (hr) | 146 | 147 | 148 | 149 | 150 | 151 | 152 | 153 | 154 | 155 | 156 | 157 | Mean | SD |
| Animal Weight | 11.1 | 10.7 | 9.5 | 8.4 | 8.0 | 9.6 | 8.3 | 9.0 | 9.5 | 8.2 | 9.7 | 9.7 | 9.3 | 1.0 |
| (kg) | | | | | | | | | | | | | | |
| Dose (mg/kg) | 18.0 | 18.7 | 21.0 | 23.8 | 25.0 | 20.8 | 24.1 | 22.2 | 21.1 | 24.4 | 20.6 | 20.6 | 21.7 | 2.2 |
| Cₘₐₓ (ng/mL) | 38.8 | 180 | 148 | 134 | 203 | 152 | 213 | 308 | 168 | 139 | 110 | ND³ | 163 | 67.4 |
| tₘₐₓ (hr) | 8.0 | 6.0 | 8.0 | 4.0 | 4.0 | 2.0 | 3.0 | 6.0 | 4.0 | 8.0 | 4.0 | ND³ | 5.2 | 2.1 |
| t_{1/2} (hr) | ND² | ND² | ND² | ND² | ND² | ND² | ND² | ND² | ND² | ND² | ND² | ND³ | ND | ND |
| MRTₗₐₛₜ (hr) | 4.73 | 4.98 | 5.18 | 4.85 | 5.00 | 4.34 | 4.75 | 5.52 | 4.79 | 4.71 | 4.58 | ND³ | 4.86 | 0.312 |
| AUCₗₐₛₜ | 181 | 932 | 647 | 619 | 651 | 424 | 861 | 1086 | 845 | 752 | 617 | ND³ | 692 | 248 |
| (hr · ng/mL) | | | | | | | | | | | | | | |
| AUC_{∞} | ND² | ND² | ND² | ND² | ND² | ND² | ND² | ND² | ND² | ND² | ND² | ND³ | ND | ND |
| (hr · ng/mL) | | | | | | | | | | | | | | |
| Dose-normalized | | | | | | | | | | | | | | |
| Values¹ | | | | | | | | | | | | | | |
| AUCₗₐₛₜ | 10.1 | 49.9 | 30.8 | 26.0 | 26.0 | 20.4 | 35.7 | 48.9 | 40.1 | 30.8 | 29.9 | ND³ | 31.7 | 11.7 |
| (hr · kg · ng/ | | | | | | | | | | | | | | |
| mL/mg) | | | | | | | | | | | | | | |
| AUC_{∞} | ND² | ND² | ND² | ND² | ND² | ND² | ND² | ND² | ND² | ND² | ND² | ND³ | ND | ND |
| (hr · kg · ng/ | | | | | | | | | | | | | | |
| mL/mg) | | | | | | | | | | | | | | |

Cₘₐₓ: Maximum plasma concentration;
tₘₐₓ: Time of maximum plasma concentration;
t_{1/2}: half-life, data points used for half-life determination are in bold in the respective plasma concentration table;
MRT: mean residence time;
AUCₗₐₛₜ: Area Under the Curve, calculated to the last observable time point;
²not determined because the terminal elimination phase was not observed;
⁴not determined due to a lack of quantifiable data points.

TABLE 5
Individual and Average Pharmacokinetic Parameters for Thiorphan After Oral Administration
of Formulation nr. 9 (example 2) in Male Beagle Dogs (200 mg Racecadotril/dog)
Formulation nr. 9 (example 2)

| | Dog # | | | | | | | | | | | | | |
|---|---|---|---|---|---|---|---|---|---|---|---|---|---|---|
| Time (hr) | 146 | 147 | 148 | 149 | 150 | 151 | 152 | 153 | 154 | 155 | 156 | 157 | Mean | SD |
| Animal Weight (kg) | 11.4 | 11.8 | 9.8 | 9.0 | 8.8 | 10.0 | 9.3 | 9.3 | 10.4 | 9.0 | 8.8 | 10.0 | 9.8 | 1.0 |
| Dose (mg/kg) | 17.5 | 16.9 | 20.4 | 22.2 | 22.7 | 20.0 | 21.5 | 21.5 | 19.2 | 22.2 | 22.7 | 20.0 | 20.6 | 1.9 |
| Cₘₐₓ (ng/mL) | 150 | 139 | 75.7 | ND⁴ | 156 | 396 | 80.8 | 232 | 101 | 251 | 260 | 153 | 181 | 95.7 |
| tₘₐₓ (hr) | 8.0 | 6.0 | 1.5 | ND⁴ | 6.0 | 3.0 | 8.0 | 6.0 | 6.0 | 2.0 | 3.0 | 3.0 | 4.8 | 2.3 |
| t_{1/2} (hr) | ND³ | ND³ | ND² | ND⁴ | ND³ | ND² | ND³ | ND³ | ND³ | ND² | 1.35 | 3.22 | 2.29 | ND |
| MRTₗₐₛₜ (hr) | 6.51 | 5.30 | 3.79 | ND⁴ | 5.57 | 4.12 | 5.32 | 5.52 | 5.54 | 4.61 | 3.57 | 4.45 | 4.94 | 0.898 |
| AUCₗₐₛₜ | 255 | 579 | 257 | ND⁴ | 548 | 933 | 277 | 805 | 445 | 978 | 878 | 520 | 589 | 273 |
| (hr · ng/mL) | | | | | | | | | | | | | | |
| AUC_{∞} | ND³ | ND³ | ND² | ND⁴ | ND³ | ND² | ND³ | ND³ | ND³ | ND² | 922 | ND⁵ | ND | ND |
| (hr · ng/mL) | | | | | | | | | | | | | | |
| Dose-normalized | | | | | | | | | | | | | | |
| Values¹ | | | | | | | | | | | | | | |
| AUCₗₐₛₜ | 14.6 | 34.3 | 12.6 | ND⁴ | 24.1 | 46.6 | 12.9 | 37.4 | 23.2 | 44.1 | 38.7 | 26.0 | 28.6 | 12.4 |
| (hr · kg · ng/ | | | | | | | | | | | | | | |
| mL/mg) | | | | | | | | | | | | | | |
| AUC_{∞} | ND³ | ND³ | ND² | ND⁴ | ND³ | ND² | ND³ | ND³ | ND³ | ND² | 40.6 | ND⁵ | ND | ND |
| (hr · kg · ng/ | | | | | | | | | | | | | | |
| mL/mg) | | | | | | | | | | | | | | |

Cₘₐₓ: Maximum plasma concentration;
tₘₐₓ: Time of maximum plasma concentration;
t_{1/2}: half-life, data points used for half-life determination are in bold in the respective plasma concentration table;
MRT: mean residence time;
AUCₗₐₛₜ: Area Under the Curve, calculated to the last observable time point;
AUC_{∞}: Area Under the Curve, extrapolated to infinity;
ND: Not Determined;
¹Dose-normalized by dividing the parameter by the dose of racecadotril in mg/kg;
²not determined because the terminal elimination phase had an R² of less than 0.85;
³not determined because the terminal elimination phase was not observed;
⁴not determined due to a lack of quantifiable data points;
⁵not determined because the AUC_{∞} was a greater than 25% extrapolation above the AUCₗₐₛₜ

### Example 5: Oral Administration Pharmacokinetic Studies in Rats

Formulations were administered to rats at doses expected to be pharmacologically effective for estimation of the bioavailability. Blood was drawn at specified time points and plasma concentration of thiorphan was assessed.

### Study Parameters:

Animals: Sprague Dawley rats, approximate weight 250 g.
Animal preparation: Deprived of food (but not of water) during 18 h preceding dosing.
Mode of administration: thiorphan (i.v.) and racecadotril (p.o.) by oral gavage.
Blood sampling volume: 300 μl/per sample.

Blood was drawn at specified time points. Plasma was immediately frozen and kept at −80° C. for assessment of thiorphan concentration by HPLC.

### Example 5A

Formulation nr. 8 (example 2) was administered to rats p.o. at dose of 20 mg/kg. One group of rats was given thiorphan i.v. at a dose of 13.4 mg/kg as a reference.

### Results:

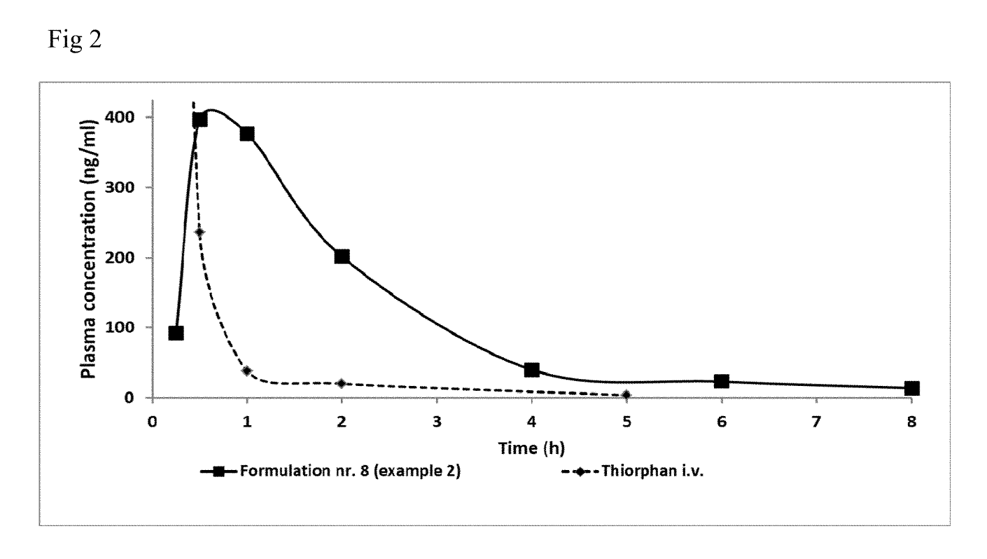

FIG. 2 and Table 6 show plasma concentration results, increased bioavailability was observed in term of AUC and Cₘₐₓ for formulation nr 8 (example 2).

TABLE 6
Individual and Average Pharmacokinetic Parameters for Thiorphan. Formulation
nr. 8 (example 2) was administered to rats p.o. at dose of 20 mg/kg. One group
of rats was given thiorphan i.v. at a dose of 13.4 mg/kg as a reference.

| | | | | | | | | Rel BA |
|---|---|---|---|---|---|---|---|---|
| | | | T_half | Tmax | Cmax | AUCt | AUCinf | (%) |
| | Treatment | Subject | (hr) | (hr) | (ng/mL) | (ng/mL * hr) | (ng/mL * hr) | % |
| IV_A | Thiorphan i.v. | 1 | 1.81 | 0.033 | 21914 | 857 | 871 | |
| | | 2 | 1.31 | 0.033 | 15924 | 2392 | 2403 | |
| | | 3 | 0.57 | 0.033 | 17649 | 1843 | 1857 | |
| | | | | | | Avg | 1710 | 100 |
| PO_S | Formulation nr. 8 (example 2) | 1 | 2.14 | 1.000 | 195 | 616 | 664 | |
| | (15% racecadotril, 18.8% Simethicone, MCT ad | 2 | 0.73 | 0.500 | 436 | 650 | 653 | |
| | q.s.) | 3 | 1.85 | 1.000 | 615 | 1118 | 1147 | |
| | | | | | | Avg | 822 | 32 |

*non-compartment analysis

### Example 5B

Formulations nr. 9 and 10 (example 2) were administered to rats p.o. at dose of 20 mg/kg. One group of rats was given reference product, a granulate powder from commercial capsule formulation, racecadotril (Vaprino100 mg®.

### Results:

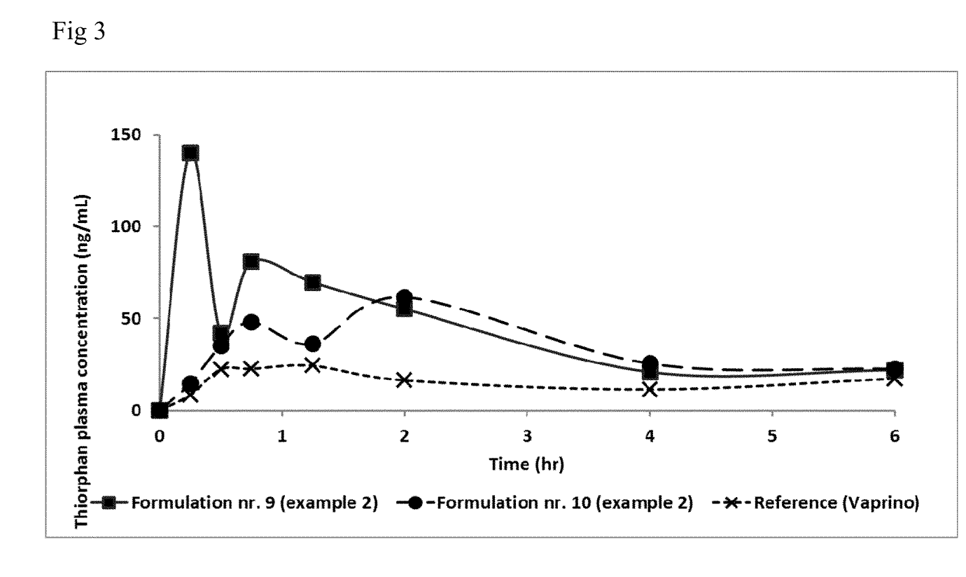

FIG. 3 shows plasma concentration results, increased bioavailability was observed in term of AUC and Cₘₐₓ for formulation nr 9 and 10 (example 2) as compared to reference.

### Example 5C

Formulations with different ratios of simethicone were tested. Formulations nr. 14, 15 and 16, (example 2) were administered to rats p.o. at dose of 20 mg/kg. One group of rats was given reference product, a granulate powder from commercial capsule formulation, racecadotril (Vaprino100 mg®).

### Results:

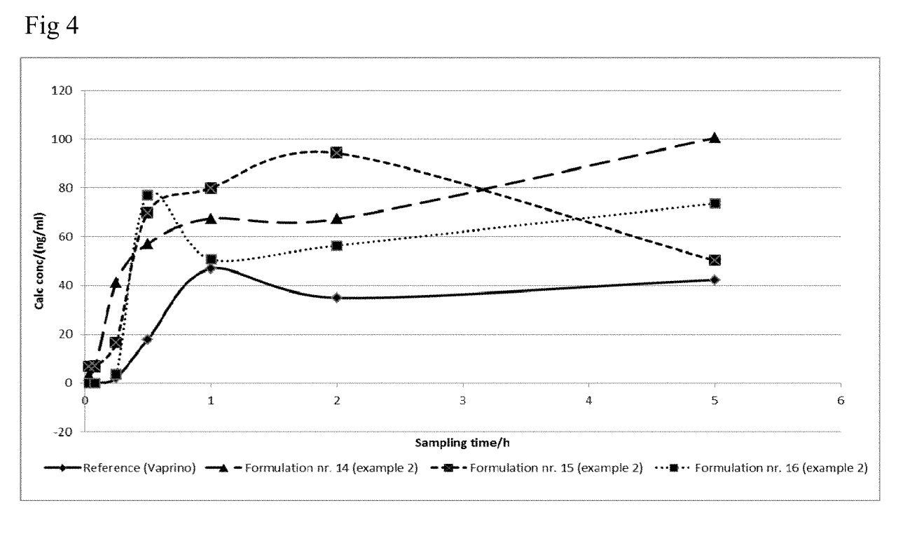

FIG. 4 shows plasma concentration results. Both immediate and prolonged release profiles were observed as well as an increased bioavailability in term of AUC and Cₘₐₓ was observed for formulations nr 14, 15 and 16 (example 2) as compared to reference.

### Example 5D

Propylene glycol based formulations of racecadotril, alone or in combination with simethicone were tested. Formulations nr. 4, and 11, (example 2) were administered to rats p.o. at dose of 20 mg/kg. One group of rats was given reference product, a granulate powder from commercial capsule formulation, racecadotril (Vaprino100 mg®.

### Results:

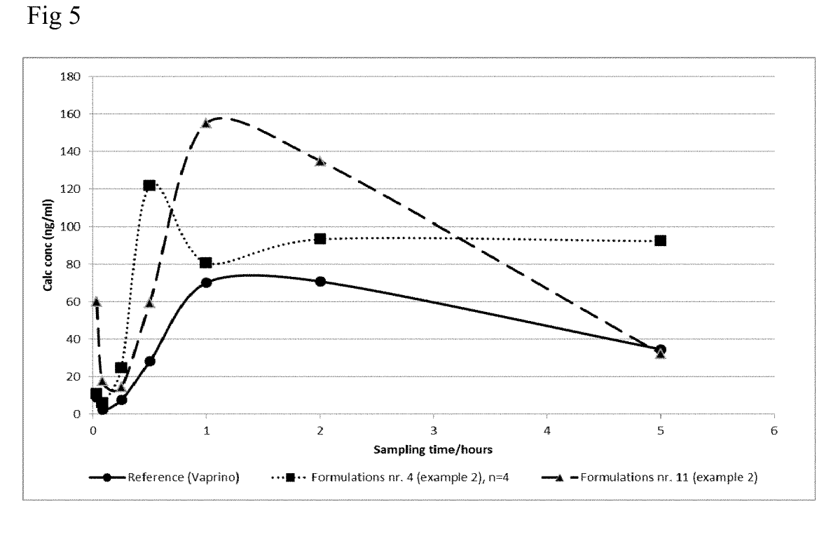

FIG. 5 shows plasma concentration results. Immediate release profile as well as increased bioavailability in term of AUC and Cₘₐₓ were observed for both formulations nr. 4, and 11, (example 2). In addition, a prolonged absorption profile was observed with formulation nr 4 (example 2) as compared to reference.

1. A semi-solid composition comprising
a. at least one cadotril and
b. at least one liquid-lipid and/or polyol and/or glycerol and/or propylene glycol excipient.

2. The semi-solid composition according to claim 1, wherein the at least one liquid-lipid excipient is at least one medium chain triglycerides or at least an oil containing medium chain triglycerides or mixtures thereof.

3. The semi-solid composition according to claim 1, wherein the composition is substantially free from non-ionic surfactants and/or is substantially free from water.

4. The semi-solid composition according to claim 2, wherein the at least one medium chain triglyceride is at least an ester of glycerol and at least one or more medium chain fatty acids selected from the group consisting of caprylic acid (C8), caproic (C6) acid, capric acid (C10), lauric acid (C12) and a mixture thereof.

5. The semi-solid composition according to claim 4, wherein the at least one medium chain triglyceride is an ester of glycerol and the one or more medium chain fatty acids is caprylic or capric acid or a mixture thereof.

6. The semi-solid composition according to claim 1, wherein the at least one cadotril coexists in a dissolved and solid state.

7. The semi-solid composition according to claim 1, wherein the at least one cadotril is selected from the group consisting of racecadotril, dexecadotril, ecadotril, and mixtures thereof.

8. The semi-solid composition according to claim 7, wherein the at least one cadotril is racecadotril.

9. The semi-solid composition according to claim 1, wherein the composition further comprises simethicone.

10. The semi-solid composition according to claim 1, wherein the at least one liquid-lipid and/or polyol and/or glycerol and/or propylene glycol excipient is/are present in an amount of from about 15% w/w to about 95% w/w of the total amount.

11. The semi-solid composition according to claim 1, wherein the at least one cadotril(s) is/are present in an amount from about 5% w/w to about 50% w/w.

12. The semi-solid composition according to claim 1, further comprising simethicone in an amount from about 5% w/w to about 75% w/w.

13. The semi-solid composition according to claim 1, wherein the composition further comprises one or more ingredient(s) selected from the list consisting of colorings, flavors, sweeteners, thickeners, emulsifiers, antioxidants, preservatives, gelling agents and disintegrants.

14. The semi-solid composition according to claim 1, wherein said composition comprises at least one dietary fibre.

15. A dose unit comprising the composition, which comprises (a) at least one cadotril; and (b) at least one liquid-lipid and/or polyol and/or glycerol and/or propylene glycol excipient.

16. The dose unit according to claim 15, wherein the at least one cadotril(s) is/are present in an amount from about 5 mg to about 200 mg.

17. The dose unit according to claim 15, wherein simethicone is present in an amount from about 50 mg to about 1500 mg.

18. A method of treatment comprising administering the semi-solid composition according to claim 1 to a subject suffering from a disease or disorder in the gastro intestinal tract.

19. A method of treatment comprising administering the dose unit according to claim 15 to a subject suffering from a disease or disorder in the gastro intestinal tract.
